(19) United States
(12) Patent Application Publication
(10) Pub. No.: US 2025/0387447 A1
(43) Pub. Date: Dec. 25, 2025

(54) Herbal composition for use in the improvement of physical and mental health recovery and maintenance, including the improvement of cognitive function or prevention of cognitive decline

(71) Applicants: Humphrey Bodington, Yatton, Hereford and Worcester (GB); Conka Elite Ltd, Yatton, Hereford and Worcester (GB)

(72) Inventors: Humphrey Bodington, Yatton (GB); Paul Chazot, Durham (GB); Karen Hind, Crawcrook (GB)

(21) Appl. No.: 18/879,009

(22) PCT Filed: Jun. 29, 2023
(86) PCT No.: PCT/GB2023/051705

§ 371 Date: Dec. 26, 2024

(30) Foreign Application Priority Data
Jun. 29, 2022 (GB) 2209547.5

(51) Int. Cl. A61K 36/81; A61K 31/202; A61K 36/16; A61K 36/41; A61K 36/45; A61K 36/53; A61K 36/67; A61K 36/9066; A61K 47/26

(52) U.S. Cl. CPC A61K 36/81; A61K 31/202; A61K 36/16; A61K 36/41; A61K 36/45; A61K 36/53; A61K 36/67; A61K 36/9066; A61K 47/26

(57) ABSTRACT

The present invention provides a novel composition comprising Ashwagandha root (Withania somnifera), Turmeric root (Curcumalonga), Lemon Balm Leaf (Melissa officinalis), Rhodiola rosea root and Bilberry fruit (Vaccinium myrtillus). The composition may be provided for use in improving physical and mental health recovery, and also for preventing cognitive decline.

The invention to which this application relates is a herbal composition for use in the improvement of physical and mental health recovery and maintenance, including the improvement of cognitive function or the prevention of cognitive decline.

Cognition or cognitive function can be affected by numerous factors such as injury, jet lag, stress, side effects from certain medications, and ageing. Thus, the possibility of improving cognitive function or preventing cognitive and mental health decline in an individual is of huge importance in today's society, in order to improve or at least maintain a desired quality of life. Herbal compositions purporting to treat a wide range of ailments are commonplace, however, such compositions are commonly an amalgamation of a number of ingredients which, individually may be known to provide some benefit. However, there is rarely any synergistic effect resulting from a specific combination of chosen ingredients.

It is therefore an aim of the present invention to provide a novel and improved herbal composition.

It is a further aim of the present invention to provide a novel and improved herbal composition for use in the improvement of physical and mental health recovery.

It is yet a further aim of the present invention to provide a novel and improved herbal composition for use in the prevention of cognitive decline.

According to a first aspect of the invention there is provided a composition comprising Ashwagandha root (Withania somnifera), Turmeric root (Curcuma longa), Lemon Balm Leaf (Melissa officinalis L.), Rhodiola rosea root and Bilberry fruit (Vaccinium myrtillus).

Typically, said composition is provided for use in improving physical and mental health recovery.

Typically, said composition is provided for use in preventing cognitive decline.

Preferably, said composition comprises Ashwagandha root (Withania somnifera), Turmeric root (Curcuma longa), Lemon Balm Leaf (Melissa officinalis), Rhodiola rosea root and Bilberry fruit (Vaccinium myrtillus) provided in a therapeutically effective amount.

In some embodiments, one or more of Ashwagandha root (Withania somnifera), Turmeric root (Curcuma longa), Lemon Balm Leaf (Melissa officinalis), Rhodiola rosea root and/or Bilberry fruit (Vaccinium myrtillus) may be provided in the form of liposomal Ashwagandha root (Withania somnifera), Turmeric root (Curcuma longa), Lemon Balm Leaf (Melissa officinalis), Rhodiola rosea root and/or Bilberry fruit (Vaccinium myrtillus).

In one embodiment, said composition further comprises an amount of Omega-3.

The present invention therefore provides a composition comprising five herbal products which individually are shown to provide various physical and mental benefits, and in combination provide a synergistic effect to optimise their properties. Typically, Ashwagandha is an anxiolytic and/or includes the properties to support muscle mass retention, anti-inflammatory qualities and/or positive effects on cognition, in particular, memory. Typically, Turmeric functions as an anti-inflammatory and/or antioxidant. Typically, Lemon Balm provides calming properties, maintaining cognitive focus and attention, and/or anti-seizure/analgesic qualities. Rhodiola rosea has been used to address the effects of mental fatigue, exercise performance and for mood elevation. Bilberry has been chosen as an anti-inflammatory and an antioxidant, and its ability to improve long-term and working memory in older adults.

In one embodiment, the composition may further include a therapeutically effective amount of Wood betony (Stachys betonica).

In one embodiment, the composition may further include therapeutically effective amount of Ginkgo (Ginkgo biloba).

In one embodiment, the composition may further include an amount of black pepper.

Typically, said composition is provided to be administered orally.

In one embodiment, the composition is provided in powder form, in capsule or tablet form, or as a powdered drink. Typically, said capsule or tablet further includes a pharmaceutically acceptable excipient.

In another embodiment, the composition is provided in liquid form. In one embodiment the liquid form includes a tincture.

Typically, the composition is provided in powder form, in capsule form, or as a powdered drink and further includes an amount of powdered Omega-3.

Typically, the composition is provided in powder form to be added to water to form a drink for consumption.

In one embodiment, the composition is provided in the form of a glycerite. Typically, a solvent forming the glycerite comprises glycerine and Tween-80 (polysorbate-80) in a ratio of glycerine: Tween-80 of 4:1. Typically, a solvent forming the glycerite is provided in a 3:1 ratio with the active components forming the composition.

In a preferred embodiment, the mass ratio of Ashwagandha: Turmeric: Lemon Balm: Rhodiola rosea: Bilberry is substantially 1:1:1:1:1.

In some embodiments, where the composition further includes an amount of Omega-3, the mass ratio of Omega-3 to Ashwagandha and Lemon Balm is 1:1.4-1.5.

In a preferred embodiment, the mass ratio of Ashwagandha:Turmeric:Lemon Balm:Rhodiola rosea:Bilberry is substantially 1:0.95:1:0.35:0.35. Further typically, the composition may be provided for oral delivery in a concentration of 1 mg/mL.

In a preferred embodiment, the composition comprises Ashwagandha root in an amount of approximately 1000 mg; Turmeric root in an amount of approximately 950 mg; Lemon Balm Leaf in an amount of 1000 mg, Rhodiola rosea root in an amount of 350 mg and Bilberry fruit in an amount of 350 mg. Typically, the composition may further include black pepper in an amount of 50 mg. Typically, the composition may further include Omega-3 in an amount of 1,400-1,500 mg.

In another aspect of the present invention, there is provided a composition for use in improving physical and mental health recovery, comprising Ashwagandha root (Withania somnifera), Turmeric root (Curcuma longa), Lemon Balm Leaf (Melissa officinalis), Rhodiola rosea root and Bilberry fruit (Vaccinium myrtillus).

Typically, said composition is provided to be taken orally in a daily dosage of Ashwagandha root in an amount of approximately 1000 mg; Turmeric root in an amount of approximately 950 mg; Lemon Balm Leaf in an amount of 1000 mg, Rhodiola rosea root in an amount of 350 mg and Bilberry fruit in an amount of 350 mg.

In one embodiment, said composition is taken in a capsule form.

In another embodiment, said composition is taken as a powdered drink.

In another embodiment, said composition is taken in tincture form, the composition being dissolved or suspended in an acceptable solvent. Typically, said composition is provided as a glycerite and a solvent forming the glycerite comprises glycerine and Tween-80 (polysorbate-80) in a ratio of glycerine:Tween-80 of 4:1. Typically, a solvent forming the glycerite is provided in a 3:1 ratio with the active components forming the composition.

In one embodiment, the composition is formed as 3.75 g of active components, made up with solvent to 11.25 mL final product.

In another aspect of the present invention, there is provided a composition for use in preventing cognitive decline, comprising Ashwagandha root (Withania somnifera), Turmeric root (Curcuma longa), Lemon Balm Leaf (Melissa officinalis), Rhodiola rosea root and Bilberry fruit (Vaccinium myrtillus).

Typically, the composition is provided to be taken orally in a daily dosage of Ashwagandha root in an amount of approximately 1000 mg; Turmeric root in an amount of approximately 950 mg; Lemon Balm Leaf in an amount of 1000 mg, Rhodiola rosea root in an amount of 350 mg and Bilberry fruit in an amount of 350 mg.

In one embodiment, said composition is taken in a capsule form.

In another embodiment, said composition is taken as a powdered drink.

In another embodiment, said composition is taken in tincture form, the composition being dissolved or suspended in an acceptable solvent. Typically, said composition is provided as a glycerite and a solvent forming the glycerite comprises glycerine and Tween-80 (polysorbate-80) in a ratio of glycerine:Tween-80 of 4:1. Typically, a solvent forming the glycerite is provided in a 3:1 ratio with the active components forming the composition.

In one embodiment, the composition is formed as 3.75 g of active components, made up with solvent to 11.25 mL final product.

Embodiments of the present invention will now be described with reference to the accompanying figures, wherein:

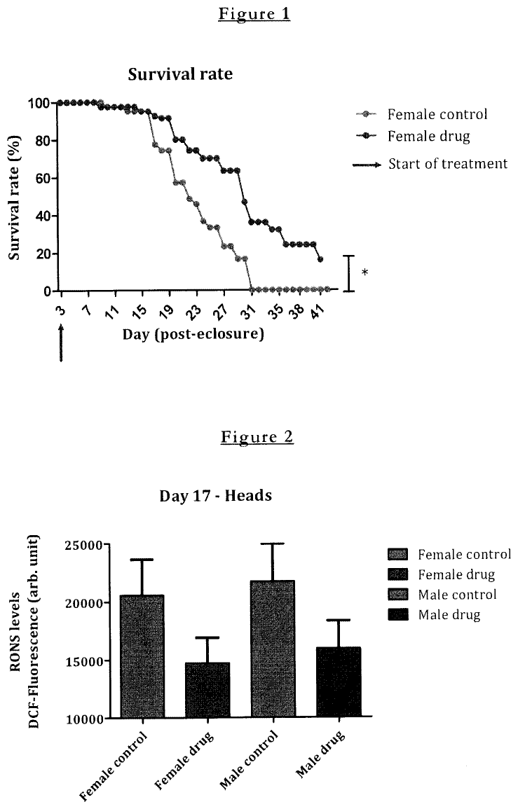

FIG. 1 illustrates the effect of treatment (oral) of a composition in accordance with an embodiment of the present invention on the survival rate of a common fruit fly (Drosophila melanogaster);

FIG. 2 illustrates the effect of treatment (oral) of a composition in accordance with an embodiment of the present invention on the oxidative stress (reactive oxygen and nitrogen species, RONS) in the brain of a common fruit fly (Drosophila melanogaster);

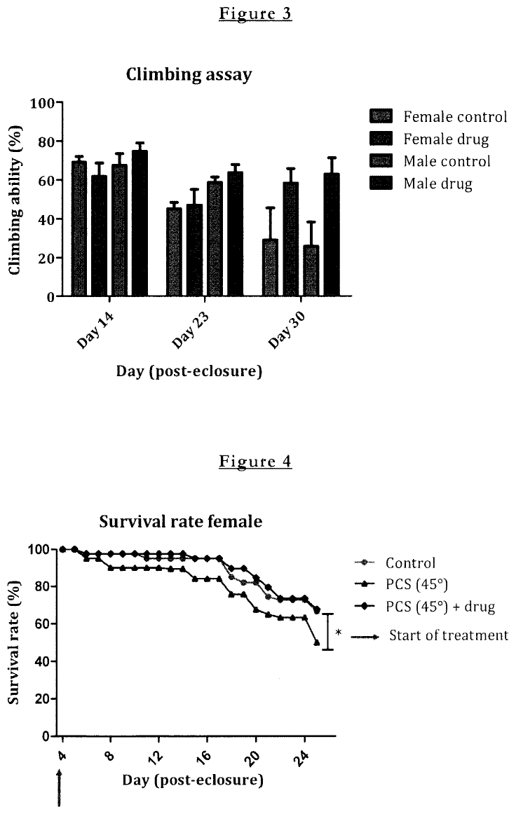

FIG. 3 illustrates the effect of treatment (oral) of a composition in accordance with an embodiment of the present invention on the mobility deficits in an ageing common fruit fly (Drosophila melanogaster); and

FIG. 4 illustrates the effect of treatment (oral) of a composition in accordance with an embodiment of the present invention on the survival rate deficit of a common fruit fly (Drosophila melanogaster) elicited by Traumatic brain injury model.

Withania somnifera, commonly called Ashwagandha, is a winter cherry tropical to the Solanaceae family. Ashwagandha contains various non-nutritional chemicals, responsible for its health-promoting properties. Regarding physical health benefits, Ashwagandha supplementation has been strongly associated with significant increases in muscle mass and strength, which suggests that Ashwagandha may be useful, in conjunction with a resistance-training program (see Examining the effect of Withania somnifera supplementation on muscle strength and recovery: a randomized controlled trial. J Int Soc Sports Nutr. 2015 Nov. 25;12:43. doi: 10.1186/s12970-015-0104-9.). A recent study also showed a significant enhancement in VO₂ₘₐₓ in healthy adults and athletes, with Ashwagandha supplementation (see Effects of Ashwagandha (Withania somnifera) on VO2max: A Systematic Review and Meta-Analysis. Nutrients. 2020 Apr. 17; 12(4): 1119. doi: 10.3390/nu12041119.). Ashwagandha root extract can also successfully enhance cardiorespiratory endurance, and can aid in attaining better physiological, metabolic, and functional abilities in humans (see A double-blind, randomized, placebo-controlled trial on the effect of Ashwagandha (Withania somnifera dunal.) root extract in improving cardiorespiratory endurance and recovery in healthy athletic adults. May 23; 272:113929. Ethnopharmacol. 2021 doi: 10.1016/j.jep.2021.113929. Epub 2021 Feb. 15.PMID: 33600918Clinical Trial.). Importantly, no adverse events were reported by any of the subjects in these studies.

Ashwagandha has also been studied for its mental health benefits. Withania somnifera root extract is neuroprotective and neuroreparative in neuronal cells, suggesting therapeutic potential to target factors involved in secondary injury and long-term sequelae of mild TBI (see Withania somnifera Extract Protects Model Neurons from In Vitro Traumatic Injury. J Neuroinflammation 2016 Aug. 22; 13(1):193). Moreover, using primary microglial cells and BV-2 microglial cell lines, the Withania somnifera root extract has been shown to inhibit microglial activation and migration, thus a potential candidate for the suppression of neuroinflammation (see Withania somnifera Extract Protects Model Neurons from In Vitro Traumatic Injury. J Neuroinflammation 2016 Aug. 22; 13(1):193 and Aqueous extract from the Withania somnifera leaves as anti-a potential neuroinflammatory agent: a mechanistic study J Neuroinflammation 13, 193 (2016).). In human studies, significant improvement was observed in both sleep parameters and anxiety indicators, with Ashwagandha root extract treatment for 10 weeks, therefore offering a potential use to improve sleep quality in athletes with insomnia and mood issues during a season (see An alternative treatment for anxiety: a systematic review of human trial results reported for the Ayurvedic herb ashwagandha (Withania somnifera). Complement Med. 2014 December; 20(12):901-8 and Efficacy and Safety of Ashwagandha (Withania somnifera) Root Extract in Insomnia and Anxiety: A Double-blind, Randomized, Placebo-controlled Study. Cureus. 2019 Sep. 28;11(9): e5797. doi: 10.7759/curcus.5797.PMID: 31728244).

Regarding Turmeric, dietary curcumin is effective at inhibiting maladaptive cardiac repair and preserving cardiac function after ischacmia and reperfusion. Daily treatment with curcumin during reperfusion protected against maladaptive tissue repair and improved cardiac function after ischaemia. Cardioprotection with curcumin was associated with attenuation of lipid peroxidation and active MMPs, inhibition of the TGFβ1-Smad signalling pathway and differentiation of fibroblasts in the balance between collagen degradation and synthesis (all linked to fibrosis). Through all these beneficial effects, curcumin could maintain the ECM integrity and stability responsible for cardiac recovery (see Curcumin promotes cardiac repair and ameliorates cardiac dysfunction following myocardial infarction. Br J Pharmacol. 2012 167(7): 1550-1562).

Several reports suggest that the well-established anti-inflammatory, antioxidant and anti-apoptotic pharmacological properties of Curcumamay be beneficial in treating brain injury. Curcumin has been shown to activate the expression of thioredoxin, an antioxidant protein in the Nrf2 pathway, which protects neurons from death caused by oxygen-glucose deprivation in an in vitro model of ischemia/reperfusion (see Curcumin pretreatment and post-treatment both improve the antioxidative ability of neurons with oxygen-glucose deprivation. Neural Regen Res. 2015; 10:481-489). Furthermore, animal models have been used to show the positive biochemical and morphological effects of the Curcuma on the prefrontal cortex (PFC) and hippocampus, which are the brain regions affected by chronic stress, and crucial for learning and memory performance. High levels of corticosterone in the hippocampus and the PFC with chronic stress cause atrophy of the dendrites of the key neurons and dendritic structural plasticity. Recent reports have demonstrated that curcumin not only increases levels of brain derived neurotrophic factor (BDNF) in the hippocampus and PFC (see Curcuma longa L. extract improves the cortical neural connectivity during the aging process Neural Regen Res. 2017 June; 12(6): 875-880; Curcumin reverses the effects of chronic stress on behavior, the HPA axis, BDNF expression and phosphorylation of CREB. Brain Res. 2006; 1122:56-64; and Effects of curcumin on learning and memory deficits, BDNF, and ERK protein expression in rats exposed to chronic unpredictable stress. Behav Brain Res. 2014; 271:116-121), but also reduces serum corticosterone levels in the animal model of chronic stress. Therefore, Curcuma can be considered as a useful tool to improve synaptic plasticity and encourage neural repair in the injured brain.

Further, there is good evidence for Melissa officinalis (Lemon Balm) having both acute anxiolytic (anti-anxiety) effects and long-term anti-agitation effects. In human studies, Lemon Balm can increase self-rated ‘calmness’ using established mood scales. Additionally, when human subjects are subjected to laboratory stressors, Lemon Balm increased calmness (see e.g. Attenuation of laboratory-induced stress in humans after acute administration of Melissa officinalis (Lemon Balm). Psychosom Med. 2004 July-August; 66(4):607-13). The mechanisms underlying the calming effects likely involve suppression hyperactive voltage-gated sodium channels (see New Insights Into the Anticonvulsant Effects of Essential Oil From Melissa officinalis L. (Lemon Balm). Front Pharmacol. 2021 Oct. 14; 12:760674). Maintaining attention and improved cognitive performance is also a feature of this product (see Modulation of mood and cognitive performance following acute administration of Melissa officinalis (lemon balm). Pharmacol Biochem Behav. 2002 July; 72(4):953-64). These mechanisms likely underpin the analgesic properties of Lemon Balm (Chazot et al., unpublished). Well-established cholinergic properties and evidence for memory enhancement are further useful properties of Lemon Balm.

Fatigue is commonly defined as a feeling of tiredness, lack of energy, emotional stability and motivation, or difficulty in concentration and memory, frequently aggravated by a variety of parallel symptoms such as headache or muscle pain, common in contact sport. Notably, repeated administration of R. rosea extract exerted an anti-fatigue effect, with reduced cortisol, together with increased mental performance, particularly the ability to concentrate (sec A randomised, double-blind, placebo-controlled, parallel-group study of the standardised extract SHR-5 of the roots of Rhodiola rosea in the treatment of subjects with stress-related fatigue. Planta Med 2009; 75:105-112). Moreover, Rhodiola extract, applied for a 4-weck period, was shown to be a safe and effective way of improving life-stress symptoms to a clinically relevant degree (see Therapeutic effects and safety of Rhodiola rosea extract WS® 1375 in subjects with life-stress symptoms—results of an open-label study. Phytother Res 2012; 26:1220-1225); effects were observed even after 3 days of treatment. In an interesting study looking at stress during an student exam period, R Rosea produced significant improvement in a series of physical fitness, mental fatigue and neuro-motoric tests (see A double-blind, placebo-controlled pilot study of the stimulating and adaptogenic effect of Rhodiola rosea SHR-5 extract on the fatigue of students caused by stress during an examination period with a repeated low-dose regimen. Phytomedicine 2000;7:85-89). In a cross-over study, healthy physicians on night duty, displayed a statistically significant improvement in general fatigue was observed in the treatment group (RRE) during the first two weeks period. The tests chosen reflect an overall level of mental fatigue, involving complex perceptive and cognitive cerebral functions, such as associative thinking, short-term memory, calculation and ability of concentration, and speed of audio-visual perception. Again, no side-effects were reported (see Rhodiola rosea in stress induced fatigue—a double blind cross-over study of a standardized extract SHR-5 with a repeated low-dose regimen on the mental performance of healthy physicians during night duty. Phytomedicine 2000; 7:365-371).

Bilberry (Vaccinium myrtillus L.) is one of the richest natural sources of anthocyanins. It has clear anti-inflammatory and lipid-lowering effects, and has been shown to lower oxidative stress, at least in the periphery. Therefore, bilberry is of potential value in the treatment or prevention of conditions associated with inflammation, including brain and peripheral injury. In mice stressed by restraint, marked increases in liver damage and reactive oxygen species (ROS) levels associated with this stress was restored to normal levels by administering a bilberry extract, together with enhanced mitochondrial function, with the bilberry treatment (sec Bilberry extract protect restraint stress-induced liver damage through attenuating mitochondrial dysfunction. Fitoterapia. 2010 December; 81(8):1094-101. doi: 10.1016/j.fitote.2010.07.004. Epub 2010 Jul. 8. Furthermore, in Vaccinium myrtillus L.-treated mice, the forelimb grip strength significantly increased, weight-loaded swimming time prolonged; their lactate, ammonia, BUN, and CK activity decreased, and muscle and liver glucose and glycogen content increased compared with the vehicle group. Thus, anthocyanins-rich plants appear to have anti-fatigue activity and an ability to increase exercise tolerance. Interestingly, bilberry supplementation reduced both soluble Aβ₄₀ and Aβ₄₂ levels compared to blackcurrant-fed mice. This suggests bilberry supplements offers a potential protective strategy for delaying long-term neurodegenerative disease (see Anthocyanin-enriched bilberry and blackcurrant extracts modulate amyloid precursor protein processing and alleviate behavioral abnormalities in the APP/PS1 mouse model of Alzheimer's disease. J Nutr Biochem. 2013;24:360-370).

Consequently, the present invention is directed to the provision of compositions comprising at least Ashwagandha root, Turmeric root, Lemon Balm Leaf, Rhodiola rosea root and Bilberry fruit. Such compositions may be provided for use in improving physical and mental health recovery, having a particular benefit for those who play contact sports such as rugby. In some embodiments of the invention, one or more of Ashwagandha root (Withania somnifera), Turmeric root (Curcuma longa), Lemon Balm Leaf (Melissa officinalis), Rhodiola rosea root and/or Bilberry fruit (Vaccinium myrtillus) may be provided in the formulation in the form of liposomal Ashwagandha root (Withania somnifera), Turmeric root (Curcuma longa), Lemon Balm Leaf (Melissa officinalis), Rhodiola rosea root and/or Bilberry fruit (Vaccinium myrtillus). By providing the active ingredients in liposomal forms in some embodiments, that is to say by containing them inside small, fat-like particles, this makes it easier and more efficient for the body to absorb and for the ingredient to take effect. The compositions may also be provided for use in improving cognitive function or preventing cognitive decline. In particular, the specific combination of the above herbal remedies was chosen owing to the synergistic effect that is created.

The composition may be provided in powder form, for consumption as a powdered drink, or included in a capsule to be taken with water or another fluid. In the provided powder form, the composition may further include an amount of powdered Omega-3. Generally, in some embodiments, the mass ratio in the composition of Ashwagandha:Turmeric:Lemon Balm Leaf:Rhodiola rosea:Bilberry is substantially 1:1:1:1:1. More specifically, in preferred embodiments, the ratio is substantially 1:0.95:1:0.35:0.35. Omega-3, where provided, is in an amount slightly higher, such that the ratio of Omega-3 to Ashwagandha or Lemon Balm Leaf is 1:1.4-1.5. Preferred amounts of the substances to be taken in a daily dose are Ashwagandha root in an amount of approximately 1000 mg; Turmeric root in an amount of approximately 950 mg; Lemon Balm Leaf in an amount of 1000 mg, Rhodiola rosea root in an amount of 350 mg and Bilberry fruit in an amount of 350 mg. Where provided, approximately 1,400-1,500 mg Omega-3may also be provided to be consumed. An example dosage formulation provides for the composition to be taken, either as a single or multiple capsules amounting to the daily dose; or a powder dissolved in water or other liquid to form a drink, to be taken three times daily. One specific embodiment may involve taking a morning dose comprising Ashwagandha, Turmeric, Lemon Balm Leaf, Rhodiola rosea root and Bilberry; an afternoon dose comprising Ashwagandha, Turmeric and Lemon Balm Leaf; and an evening dose comprising Ashwagandha, Turmeric, Lemon Balm Leaf and Omega-3. In other preferred embodiments of the invention, each of the three daily doses may be consistent and include Ashwagandha, Turmeric, Lemon Balm Leaf, Rhodiola rosea root and Bilberry, with a further option to take Omega-3 with one or more of said doses. In other embodiments of the invention, the composition may be varied further to include an amount of Wood betony (Stachys betonica) and/or Ginkgo (Ginkgo biloba). In some preferred embodiments, the composition may further include an amount of black pepper in an amount of approximately 50 mg.

The composition of the present invention may also be provided in tincture form, wherein each of the Ashwagandha root, Turmeric root, Lemon Balm Leaf, Rhodiola rosea root and Bilberry fruit are provided in therapeutically effective amounts in the formulation. In some embodiments, the composition is provided in the form of a glycerite. A typical solvent forming the glycerite comprises glycerine and Tween-80 (polysorbate-80) in a ratio of glycerine:Tween-80 of 4:1, and the solvent itself is provided in a 3:1 ratio with the active components forming the composition. Typically, the composition is formed as 3.75 g of active components, made up with solvent to 11.25 mL final product.

A summary of pilot data (Fraysse A, Hind K, Chazot P 2022; currently unpublished) obtained for the present invention is discussed: the invention was initially tested on Fruit Flies (Drosophila melanogaster) in a classic in vivo ageing model (oral delivery in the feed (1 mg/ml), Ashwagandha root, Turmeric root, Lemon Balm leaf, Rhodiola rosearoot, Bilberry (ratio: 1:1:1:0.35:0.35). It was found that the invention increases life span (FIG. 1), reduces reactive oxygen and nitrogen species in the brain (FIG. 2), and improves mobility deficits in the ageing fly (FIG. 3). Notably, the treatment reversed the survival deficits elicited by a concussion model in the ageing fly (FIG. 4).

A subsequent series of experiments were carried out using the Drosophila melanogaster fly model were performed and which confirmed the synergistic effect of the combination of Ashwagandha root, Turmeric root, Lemon Balm leaf, Rhodiola rosea root and Bilberry and the positive effects of compositions comprising those substances on ageing, motor activity and oxidative stress in normal life, and when repetitive concussions were experienced in early life using a novel validated model (Sports Post-Concussion Syndrome (PCS) Model in Drosophila melanogaster. English & Chazot (2021) Alzheimer's & Dementia Supplement: Basic science 17 (2).

### Lifespan Extension

Compositions comprising the five active substances were tested and displayed a significant extension of lifespan. In contrast, when one component was removed, this life extension was lost, apart from when Ashwaganda was removed (p<0.05).

### Health Span Based on Climbing Motor Assay

Compositions comprising the five active substances were tested and displayed a significant improvement of motor activity at days 31-33 (human equivalent age ca. 62). In contrast, when one component was removed, this extension was lost in all cases, apart from when Rhodiola was removed (*p<0.05)

### RONS Activity in Head (Brain) and Body

Compositions comprising the five active substances were tested and displayed a significant reduction in Reactive oxidative and nitrogen species (RONS) activity (oxidative stress) at day 31. When one component was removed, this reduction in RONS (anti-oxidant) was lost in all cases for the brain samples, apart from when Lemon Balm leaf and turmeric were removed in the body sample, respectively (*p<0.05).

### RONS activity in head and body (age 31) following 5 repetitive concussions (PCS, Post-concussion samples)

Compositions comprising the five active substances were tested and displayed a significant reduction in RONS activity (oxidative stress) (anti-oxidant effect) after the repetitive concussions. Ashwaganda (body), Melissa (brain) and turmeric (body) are required for protection against oxidative stress (RONS) caused by repetitive concussions. In the absence of the other components, the inhibition of oxidative stress was still observed (*p<0.05).

All of Ashwagandha root, Turmeric root, Lemon Balm leaf, Rhodiola rosea root and Bilberry are required for the complete set of positive effects observed with the composition of the present invention, that is to say, life span extension, motor activity and brain and body RONS (oxidative stress) with and without repetitive concussions experienced earlier in life.

1. A composition comprising Ashwagandha root (Withania somnifera), Turmeric root (Curcuma longa), Lemon Balm Leaf (Melissa officinalis), Rhodiola rosea root and Bilberry fruit (Vaccinium myrtillus).

2. A composition according to claim 1, wherein said composition is provided for use in improving physical and mental health recovery.

3. A composition according to claim 1, wherein said composition is provided for use in preventing cognitive decline.

4. (canceled)

5. A composition according to claim 1, wherein said composition further comprises an amount of Omega-3.

6. A composition according to claim 1, wherein the composition further includes an amount of Wood betony (Stachys betonica) and/or Ginkgo (Ginkgo biloba).

7. A composition according to claim 1, wherein the composition further includes an amount of black pepper.

8. A composition according to claim 1, wherein the composition is provided in powder form, in capsule or tablet form, or as a powdered drink.

9. A composition according to claim 1, wherein the composition is provided in liquid form, and includes a tincture.

10. A composition according to claim 1, wherein the composition is provided in powder form to be added to water to form a drink for consumption.

11. A composition according to claim 1, wherein the composition is provided in the form of a glycerite.

12. A composition according to claim 11, wherein a solvent forming the glycerite comprises glycerine and Tween-80 (polysorbate-80) in a ratio of glycerine:Tween-80 of 4:1.

13. A composition according to claim 11, wherein a solvent forming the glycerite is provided in a 3:1 ratio with the active components forming the composition.

14. A composition according to claim 1, wherein the mass ratio of Ashwagandha root:Turmeric root:Lemon Balm Leaf:Rhodiola rosea root:Bilberry fruit is substantially 1:1:1:1:1.

15. A composition according to claim 1, wherein the mass ratio of Ashwagandha root:Turmeric root:Lemon Balm Leaf:Rhodiola rosea root:Bilberry fruit is substantially 1:0.95:1:0.35:0.35.

16. A composition according to claim 5, wherein the mass ratio of Omega-3 to Ashwagandha root and/or Lemon Balm Leaf is substantially 1:1.4-1.5.

17. A composition according to claim 1, wherein the composition comprises Ashwagandha root in an amount of approximately 1000 mg; Turmeric root in an amount of approximately 950 mg; Lemon Balm Leaf in an amount of 1000 mg, Rhodiola rosea root in an amount of 350 mg and Bilberry fruit in an amount of 350 mg.

18. (canceled)

19. (canceled)

20. A composition for use in improving physical and mental health recovery, comprising Ashwagandha root (Withania somnifera), Turmeric root (Curcuma longa), Lemon Balm Leaf (Melissa officinalis), Rhodiola rosea root and Bilberry fruit (Vaccinium myrtillus).

21. A composition for use according to claim 20, wherein the composition is provided to be taken orally in a daily dosage of Ashwagandha root in an amount of approximately 1000 mg; Turmeric root in an amount of approximately 950 mg; Lemon Balm Leaf in an amount of 1000 mg, Rhodiola rosea root in an amount of 350 mg and Bilberry fruit in an amount of 350 mg.

22. A composition for use in preventing cognitive decline, comprising Ashwagandha root (Withania somnifera), Turmeric root (Curcuma longa), Lemon Balm Leaf (Melissa officinalis), Rhodiola rosea root and Bilberry fruit (Vaccinium myrtillus).

23. A composition for use according to claim 22, wherein the composition is provided to be taken orally in a daily dosage of Ashwagandha root in an amount of approximately 1000 mg; Turmeric root in an amount of approximately 950 mg; Lemon Balm Leaf in an amount of 1000 mg, Rhodiola rosea root in an amount of 350 mg and Bilberry fruit in an amount of 350 mg.
